(19) United States
(12) Patent Application Publication
(10) Pub. No.: US 2003/0044766 A1
(43) Pub. Date: Mar. 6, 2003

(54) Methods and devices for detecting cell-cell interactions

(76) Inventors: Anne Scholz, Santa Cruz, CA (US); Martin Bonde, San Jose, CA (US); Steven Kain, San Carlos, CA (US)

Correspondence Address: REED & ASSOCIATES, 800 MENLO AVENUE, SUITE 210, MENLO PARK, CA, 94025, US

(21) Appl. No.: 09/942,534

(22) Filed: Aug. 29, 2001

(51) Int. Cl. C12Q 1/00; C12Q 1/04; G01N 1/30; G01N 33/48

(52) U.S. Cl. 435/4; 435/34; 435/40.5

(57) ABSTRACT

The invention relates to methods and devices for detecting a cell-cell interaction between a first living cell and a second living cell. The method comprises (a) providing a flow passage defined at least in part by a substrate having a first living cell, or tissue section containing living cells, immobilized on a surface thereof, (b) introducing a second living cell by controlled delivery of a carrier fluid containing the second living cell in contiguous laminar flow through the flow passage, thereby effecting contact or proximity between the first living cell and the second living cell, and (c) detecting a cell-cell interaction, if present, as a result of the contact or proximity between the first living cell and the second living cell. Devices for carrying out the method and for providing a cell-cell interaction are provided as well.

### TECHNICAL FIELD

The present invention relates to methods and devices for conducting assays to determine cell-cell interactions. More specifically, the invention relates to methods and devices for detecting cell-cell interactions based on directing a cell-containing fluid over a substrate having living cells or tissue comprising living cells immobilized thereto.

### BACKGROUND

Biological cells represent the primary building blocks of higher biological systems, such as tissues and organs, as well as entire multicellular organisms. In higher organisms, e.g., mammals, cells must often interact with each other for such purposes as transmitting signals and building macrostructures, including tissues. Because cell interactions may influence disease states, such as autoimmune disorders, atherosclerosis, psoriasis, or metastatic cancers, scientists have been greatly interested in studying them.

To this end, several approaches have been suggested for studying cell-cell interactions.

Early in vitro models were based on the so-called Stamper-Woodruff assay. In this assay, a suspension of lymphocytes is placed on top of a thin section of rat or mouse tissue. The force of gravity brings the lymphocytes in contact with the tissue section. Once contact has been established, bound cells are fixed, visualized, and identified under light microscope. See Stamper et al. (1976) J Exp Med 144(3):828-833.

Variations on the Stamper-Woodruff assay have also been tried. See, for example, U.S. Pat. No. 6,010,845 to Poston. A significant drawback to these approaches is that they involve the use of relatively large amounts of cells. Furthermore, the Stamper-Woodruff assay format does not mimic physiological conditions, relying instead upon gravity or centrifugal forces (as opposed to other significant influences commonly exhibited in vivo, such as fluid flow). Also, some cells may not be easily visualized using standard light microscopy techniques.

U.S. Pat. No. 5,656,441 to Faller et al. describes the labeling of cells, followed by detection of their label signals, in order to better identify them. This process, however, requires the extra step of labeling the cells, which could potentially interfere with the specific cell-cell interactions being tested.

Microtiter plates have also been used to study cell-cell interactions. Often, a washing step is required when microtiter plates are employed for such studies. Conducting this washing step in an effort to preserve a specific cell-cell interaction and to observe this interaction under physiological conditions of fluid shear stress, however, is extremely difficult. Such difficulty may stem from the fact that the shear forces associated with the washing process are much greater than the binding forces (if any) that are present during cell-cell interactions. As the forces associated with antibody binding, for example, are much greater than those associated with more rudimentary cell-cell interactions, washing steps are, in effect, less disruptive to microtiter-based assays, such as those involving antibody binding, than they are to the detection of cell-cell interactions in a microtiter plate.

In addition, conventional microtiter wells only allow for the study of static binding. In contrast, biological cells are often transmitted throughout the bloodstream, wherein fluid flow is exhibited. Some early binding events that mediate recruitment of blood-borne cells from the vascular system require fluid shear stress to be active. Thus, studies involving microtiter wells lack the ability to take into account the important influence of flow conditions. As cell-cell interactions may be significantly different under static, rather than dynamic, conditions, alternative methods to those based on a microtiter well format are needed.

Some of the problems associated with conventional microtiter well-based assays have been addressed with video microscopy using commercially available flow chambers. One advantage of this method is that the reliance on labeled cells is reduced, as the cells are viewed directly.

Conventional flow chambers, however, have drawbacks of their own. For example, the throughput of assays is very slow. Additionally, the flow is relatively uncontrolled, thereby not entirely mimicking physiological conditions. Also, flow chambers associated with these methods are relatively large, i.e., about 25 mm in diameter. Such large chambers require relatively great quantities of cells and other materials in order for the desired assay to be conducted. In particular, for drug discovery applications, flow chambers often consume large amounts of drug. Consequently, flow chambers do not represent the ideal method for conducting assays that are designed to detect cell-cell interactions.

Thus, there is a need for alternative methods and devices that are capable of conducting assays to test for cell-cell interactions, optionally in the presence of a reagent. Such a method and device should allow for the ability to conduct high-throughput screening without requiring the great expense and use of materials commonly seen with conventional methods. The method and device should mimic physiological conditions so as to provide a better in vivo model. These and other advantages are provided in accordance with the present invention.

### SUMMARY OF THE INVENTION

Accordingly, it is an object of the present invention to overcome the above-mentioned disadvantages of the prior art by providing methods for detecting a cell-cell interaction comprising: (a) providing a flow passage defined at least in part by a substrate having a first living cell immobilized on a surface thereof; (b) introducing a second living cell by controlled delivery of a carrier fluid containing the second living cell in contiguous laminar flow through the flow passage, thereby effecting contact or proximity between the first living cell and the second living cell; and (c) detecting a cell-cell interaction, if present, as a result of the contact or proximity between the first living cell and the second living cell.

It is another object of the invention to provide such a method wherein a reagent is introduced before, after, or simultaneously with step (b).

It is still another object of the invention to provide such a method wherein the reagent is a small drug molecule, amino acid, amino acid analog, peptide, protein, nucleotide, nucleoside, oligonucleotide, antibody, or conjugate thereof.

It is still yet another object of the invention to provide such a method wherein the carrier fluid comprises a medium appropriate to sustain living cells.

It is still a further object of the invention to provide controlled delivery of a carrier fluid using hydrodynamically focused flow.

It is still yet another object of the invention to provide a device for providing a cell-cell interaction, wherein the device comprises: (a) a substrate having a first living cell immobilized on a surface thereof; (b) at least one inlet for introducing a carrier fluid containing a second living cell; (c) a means for controlling delivery of the carrier fluid in contiguous laminar flow so as to enable contact or proximity between the first living cell and the second living cell; and (d) at least one outlet enabling removal of fluid from the device.

It is a farther objection of the invention to provide such a device that includes a means for detecting a cell-cell interaction, if present, resulting from the contact or proximity between the first living cell and the second living cell.

Additional objects, advantages, and novel features of the invention will be set forth in part in the description that follows, and in part will become apparent to those skilled in the art upon examination of the following, or may be learned through routine experimentation upon practice of the invention.

In one embodiment, the invention provides a method for detecting a cell-cell interaction. In a first step of the method, a flow passage is provided defined at least in part by a substrate having a first living cell immobilized on a surface thereof. The first living cell may be immobilized onto the substrate surface before the substrate becomes a part of the flow passage or the first living cell may be immobilized subsequent to forming the flow passage. For example, the substrate surface may be the surface of a disposable glass slide that forms part of the flow passage. The first living cell may be immobilized onto the surface of the glass slide prior to being placed in the flow passage. In addition, the first living cell may be introduced into the flow passage after the glass slide has been inserted into the flow passage in a manner similar to the method for introducing the second living cell; this method will be explained in more detail below.

The next step comprises (b) introducing a second living cell by controlled delivery of a carrier fluid containing the second living cell in laminar flow through the flow passage, thereby effecting contact or proximity between the first living cell and the second living cell. The third step comprises (c) detecting a cell-cell interaction, if present, as a result of the contact or proximity between the first living cell and the second living cell.

In the context of the present invention, the “first living cell” may comprise a single, isolated, a plurality of living cells or a part of a tissue section containing living cells.

There are a number of different techniques that can be employed to carry out the method.

In another embodiment, the invention provides a device for carrying out the method and/or for providing contact or proximity between two cells. The device comprises (a) a substrate having a first living cell immobilized on a surface thereof; (b) at least one inlet for introducing a carrier fluid containing a second living cell; (c) a means for controlling delivery of the carrier fluid in contiguous laminar flow so as to enable contact or proximity between the first living cell and the second living cell; and (d) at least one outlet enabling removal of fluid from the device. The device may further comprise a means for detecting a cell-cell interaction when one is present.

### BRIEF DESCRIPTION OF THE DRAWINGS

FIG. 1 illustrates a device in exploded view schematically showing many features of the device as described herein.

FIGS. 2A-2D, collectively referred to as FIG. 2, illustrate a preferred device that may be employed to carry out the inventive method by directing a hydrodynamically focused stream over a target region of a substrate surface. FIG. 2A illustrates the device in exploded view.

FIGS. 2B-2D schematically illustrate the device in its assembled form, wherein a lane formed from a hydrodynamically focused stream is swept over a target region of a substrate surface from one side wall to an opposing side wall. The lane comprises a carrier fluid and a second living cell.

FIG. 3 is a chart showing the results of Example 2, demonstrating the strength of adherence of Jurkat cells to TNF-treated HUVECs (human endothelial cells) under various flow rates.

FIG. 4 is a chart showing the results of Example 3, which demonstrates the binding strength and specificity of Jurkat cells to TNF-treated and -untreated HUVECs.

FIG. 5 is a chart showing the results of Example 4, demonstrating the binding strength and specificity of Jurkat cells to HUVECs treated with various concentrations of TNF.

FIGS. 6A-6C are pictures of the results of the experiments performed in Example 5.

### DETAILED DESCRIPTION OF THE INVENTION

Before the invention is described in detail, it is to be understood that, unless otherwise indicated, this invention is not limited to particular materials, components, or manufacturing processes, as such may vary. It is also to be understood that the terminology used herein is for purposes of describing particular embodiments only, and is not intended to be limiting.

It must be noted that, as used in the specification and the appended claims, the singular forms “a,” “an,” and “the” include plural referents unless the context clearly dictates otherwise.

Thus, for example, reference to a “living cell” includes a single living cell as well as a plurality of living cells, either the same (e.g., as obtained in a tissue section) or different, reference to “an inlet” includes a single inlet as well as a plurality of inlets, and the like.

In this specification and in the claims that follow, reference will be made to a number of terms that shall be defined to have the following meanings:

The term “cell line” as used herein refers to a permanently established cell culture that will proliferate indefinitely given appropriate fresh medium and space. While cell lines are readily available for some species, such as those in the rodent family, and difficult to establish for other species, such as humans, the term “cell line” as used herein is not limited to any particular species or cell type.

The term “tissue section” is defined as a thin section of living material that contains viable cells, some of which are accessible to fluid directed over one surface of the section using laminar flow.

The term “fluid-tight” is used herein to describe the spatial relationship between two solid surfaces in physical contact, such that fluid is prevented from flowing into the interface between the surfaces.

The terms “immobilize,” “immobilized,” and “immobilizing,” e.g., as in “immobilized cells,” are used herein to describe the fixation of a cell to a position on a substrate surface.

The term “laminar flow” as used herein refers to fluid movement in the absence of turbulence. The Reynolds number associated with laminar flow described herein is typically about 0.1 to about 200, preferably about 1 to 20, and optimally about 2 to 10.

The term “lane” as used herein refers to one of a set of typically parallel and linear routes, or courses, along which a fluid travels or moves.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “primary cells” is used herein in its ordinary sense and refers to cells taken directly from a living tissue, i.e., one that has not been immortalized. Primary cells may be derived from a number of sources, such as from an in vivo or ex vivo organ culture. For example, primary cells may be taken from a liver biopsy, a fetus, or embryonic tissue.

The term “reagent” is used herein to refer to any substance that exerts or may exert an influence on a cell-cell interaction. Thus, for example, the reagent may be a drug, a drug candidate, a pharmaceutical excipient, a pharmaceutical excipient-candidate, or a model compound. Typically, the reagent will be a small drug molecule, amino acid, amino acid analog, peptide, protein, nucleotide, nucleoside, oligonucleotide, antibody, or a conjugate thereof.

The term “substrate” as used herein refers to any material having a surface over which laminar fluid flow may occur. The substrate may be constructed in any of a number of forms, such as wafers, slides, well plates, and membranes. Suitable substrate materials include, but are not limited to, supports that are typically used for cell handling such as: polymeric materials (e.g., polystyrene, polyvinyl acetate, polyvinyl chloride, polyvinyl fluoride, polyacrylonitrile, polyacrylamide, polymethyl methacrylate, polytetrafluoroethylene, polyethylene, polypropylene, polybutylene, polyvinylidene fluoride, polycarbonate, polyimide, and polyethylene teraphthalate); silica and silica-based materials; functionalized glasses; ceramics; and substrates treated with surface coatings, polymeric, and/or metallic compounds, or the like. While the foregoing support materials are representative of conventionally used substrates, it is to be understood that the substrate may in fact comprise any biological, nonbiological, organic, and/or inorganic material, and may further have any desired shape, such as a disc, square, sphere, circle, etc. The substrate surface is typically, but not necessarily, planar or flat. The substrate surface may contain, however, raised or depressed regions.

The term “surface modification” as used herein refers to the chemical, biological, and/or physical alteration of a surface by an additive or subtractive process to change one or more chemical and/or physical properties of a substrate surface or a selected location or region of a substrate surface. For example, surface modification may involve (1) changing the wetting properties of a surface; (2) functionalizing a surface, i.e., providing, modifying, or substituting surface functional groups; (3) defunctionalizing a surface, i.e., removing surface functional groups; (4) otherwise altering the chemical composition of a surface, e.g., through etching; (5) increasing or decreasing surface roughness; (6) providing a coating on a surface, e.g., a coating that exhibits wetting properties that are different from the wetting properties of the surface; and/or (7) depositing particulates on a surface. Thus, for example, surface modification may involve providing a biologically derived coating on a surface, wherein the coating comprises a naturally occurring polymer, such as a protein or peptide (e.g., collagen, fibronectin, albumin, fibrinogen, or thrombin); a saccharide (such as polymannuronic acid, polygalacturonic acid, dextran, or glycosaminoglycan); or a synthetic polymer (such as polyvinyl alcohol, acrylic acid polymers, or acrylic acid copolymers).

The term “target region” as used herein refers to a predefined two-dimensional area over which fluid is directed to flow. The target region is typically, but not necessarily, contiguous. The target region may exhibit any of a variety of surface properties as long as the surface properties are predetermined.

Thus, the invention generally relates to methods and devices for detecting cell-cell interactions. Although not limited with respect to the particular arrangement used, the methods and devices provide the ability to conduct cell-cell interaction assays in a format that mimics physiological conditions, reduces the amount of cells necessary to carry out the assay, and provides for higher throughput in comparison to conventional methods and devices.

Generally, the method involves, and the device provides for, controlled delivery of a fluid containing a second living cell (so as to effect contact or proximity between the second living cell and a first living cell or tissue section containing living cells), such that the fluid is maintained in laminar flow through a flow passage.

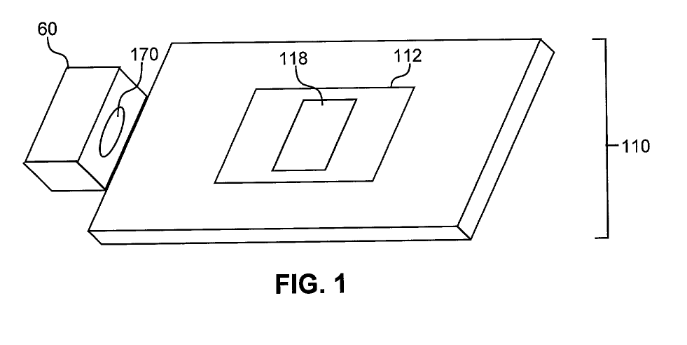

As illustrated in FIG. 1, the device 110 includes a substrate 112. Although not limited with respect to shape or material, the substrate 112 included in device 110 is preferably an ordinary glass slide, e.g., a 25 mm×75 mm glass slide or a 50 mm×75 mm glass slide. The substrate 112 represents at least a portion of a flow passage (not identified) when flow is initiated over the substrate 112. While FIG. 1 illustrates that a rectangular-shaped target region 118 is located at the center of a surface of substrate 112, the target region 118 may be any of size or shape and may be located on most any portion of the substrate 112. The surface area of the target region 118 is typically 1 mm² to about 100 mm², preferably about 10 mm² to about 50 mm², and optimally about 20 mm² to about 30 mm². Advantageously, target regions of this size reduce the total number of cells necessary to conduct a cell-cell interaction assay and/or the volume of any reagent that may be added when carrying out the assay. The target region 118 on the surface of substrate 112 has a first living cell (not shown) immobilized thereto.

The device 110 also comprises at least one inlet 170 for introducing a carrier fluid (not shown) containing a second living cell (also not shown) into the device 110, thereby allowing for the carrier fluid to contact the substrate surface and to travel through the flow passage once flow is initiated.

The device 110 also comprises a controlled delivery means 60 for delivering the carrier fluid in a controlled and directed manner over the substrate 112. Any effective controlled delivery means, e.g., pump, may be used to deliver the carrier fluid in a controlled and directed manner, such that flow is maintained under substantially laminar conditions. In operation, the controlled delivery means 60 introduces a second living cell suspended in the carrier fluid into and through the flow passage. In this way, contact or proximity between the first living cell and the second living cell is accomplished.

A preferred technique for effecting controlled delivery of a fluid containing a second living cell involves directing a hydrodynamically focused stream of fluid that contains the cell over the target region. Use of hydrodynamic focused streams in cellular assays has been described, for example, in U.S. Ser. No. 09/896,484 (“Flow Cell Assemblies and Methods of Spatially Directed Interaction Between Liquids and Solid Surfaces”), inventors Martin Bonde and Thomas Ahl, filed on Jun. 29, 2001; and aspects of hydrodynamic focusing described in that application may be employed in the present invention as well.

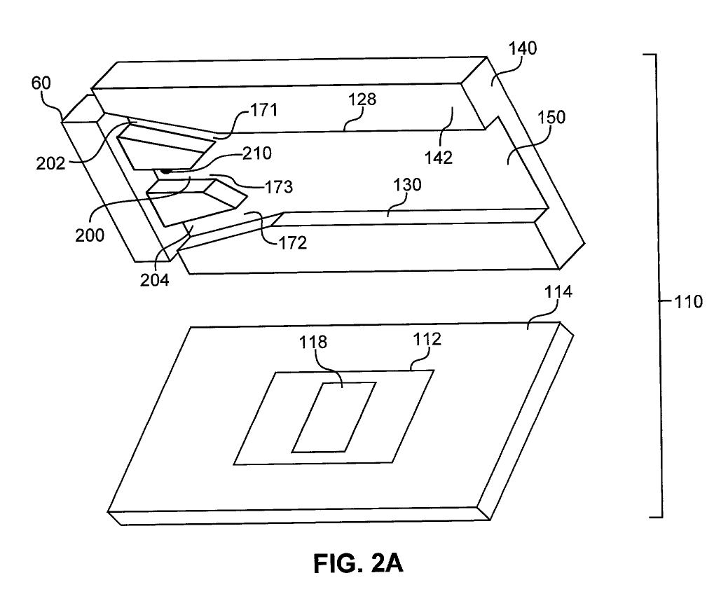

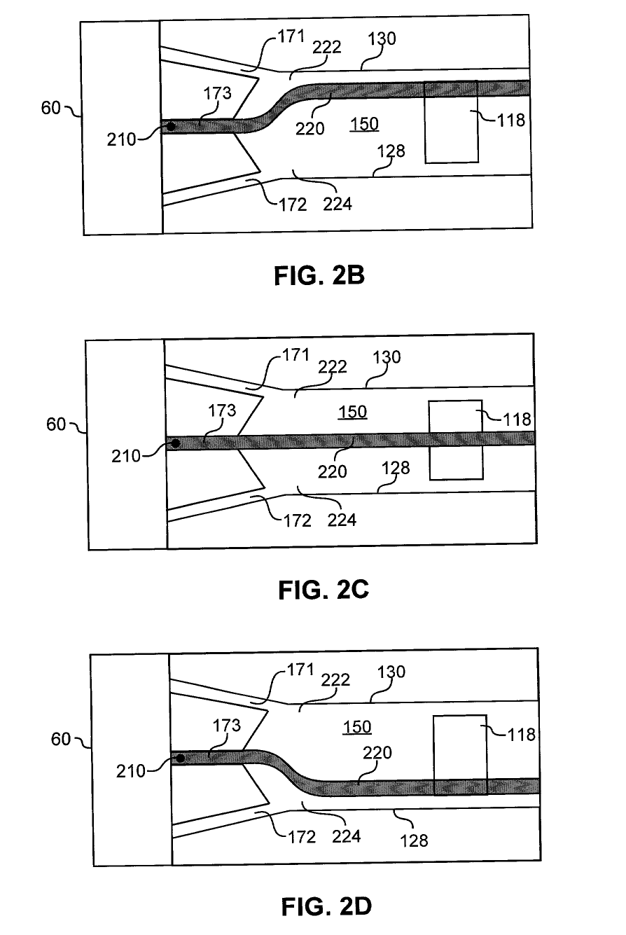

FIG. 2A illustrates a device that may be employed to provide controlled delivery, wherein this controlled delivery is effected by directing a hydrodynamically focused stream over the target region. At least three introduction channels are provided in connection with the controlled delivery means 60. That is, cell-containing stream channel 200, which includes the second living cell, is provided between two guide stream channels, indicated at 202 and 204, on an optional cover plate contact surface, such that when the cover plate contact surface is placed in contact with substrate surface 114, channels 200, 202, and 204 form introduction conduits each having an inlet indicated at 173, 171, and 172, respectively, through which fluid external to the microdevice may flow, emptying into the main flow passage 150. As shown, guide stream inlets 171 and 172 are located at the most upstream position on sidewalls 128 and 130.

In operation, as illustrated in FIGS. 2B, 2C, and 2D, the device is assembled to form the main flow passage 150 defined by the substrate, the side walls 128 and 130, and the ceiling of the main channel. The target region 118 is located within the main flow passage 150, downstream from the introduction conduits and associated inlets 173, 171, and 172. In this approach for providing controlled delivery, the controlled delivery means 60 provides guide stream inlets in fluid communication with a guide fluid source and optional reagent inlet 210. When fluid flow is provided from the sources and through inlets 173, 171, and 172, a lane 220, containing the second living cell, is formed between the two lanes 222 and 224 of guide fluids. Generally, the width of the lane containing the second living cell can be expressed as a function of the volumetric flow rate of the fluid in the lane that contains the second living cell and the flow rate of the guide streams. That is, a low flow rate of a second living cell-containing fluid in combination with a high guide stream flow rate tends to result in a narrow lane that contains the second living cell. Conversely, a high flow rate of a second living cell-containing fluid in combination with a low guide stream flow rate tends to result in a wide lane that contains the second living cell.

In addition, the position of the lane containing the second living cell depends on the relative flow rate of the fluids in each guide lane. For example, FIG. 2B illustrates the position of the second living cell-containing lane when the volumetric flow rate of the fluid in lane 224 is substantially greater than that of the fluid in lane 222. FIG. 2C illustrates the position of the second living cell-containing lane when the volumetric flow rates of the fluids in lanes 222 and 224 are approximately equal. FIG. 2D illustrates the position of the second living cell-containing lane when the volumetric flow rate of the fluid in lane 224 is substantially lower than that of the fluid in lane 222. It should be evident, then, that it is possible to direct the hydrodynamically focused second living cell-containing stream 220 from side wall 130 to side wall 128, thereby ensuring that the second living cell-containing stream 220 can be directed over any desired point of the target region 118. This is accomplished by increasing the flow rate of fluid in lane 222 to the flow rate of fluid in lane 224. Similarly, a hydrodynamically focused stream of a second living cell-containing fluid may be directed from sidewall 128 to sidewall 130, and over any point of the target region 118, by increasing the flow rate of fluid in lane 224 to the flow rate of fluid in lane 222.

A significant advantage of hydrodynamically focused flow, as well as laminar flow, as described herein, is that the fluid dynamics more closely approximates those of actual physiological conditions. Physiological conditions include those conditions found in a living organism possessing a circulatory system, preferably a mammal. Typically, such conditions include a pH of about 7.4, a temperature of about 37° C., and a fluids having a tonicity equivalent to normal saline (i.e., a 0.9% NaCl solution). In addition, physiological conditions include the phenomenon of fluid flow, particularly in a stream, due to, for example, the circulatory system of the organism. In particular, hydrodynamically focused flow mimics fluid flowing through a tube. In the context of higher organisms, this provides a model for fluid flowing through the body, e.g., blood flowing through a vein, artery, or capillary.

As previously mentioned, an optional reagent may be introduced before, after, or along with the introduction of the second living cell. When the reagent is introduced before the introduction of the second living cell, for example, a pump may be used to draw fluid from a vessel containing only the reagent, which is then introduced into the flow passage. Thereafter, the same pump may draw fluid from a different vessel that contains only a suspension of cells. In this way, the reagent and cells are sequentially introduced into the flow passage. Reversing this order, of course, results in the introduction of the cell prior to introduction of the reagent. Simultaneous introduction of the optional reagent and the cell can be accomplished by addition of the reagent to the cell-containing suspension from which the pump draws. Dedicated reagent inlets and pumps may also be used, wherein introduction of the cell and reagent is timed in order to provide the desired introduction order.

A particularly preferred method for introducing the living cell and/or reagent includes providing a fluid vessel having a cavity extending from an inlet opening to an outlet opening and loading the reagent, a plurality of different reagents (if desired), or the same reagent at different concentrations. The living cell, reagent, or reagents may then be released (sequentially, if more than one cell and/or reagent is loaded) through the inlet opening to the outlet opening, thereby being subsequently discharged into the flow passage. The sequence is selected to correspond to the desired sequence with which the living cell and/or reagent will be released. For small volumes of fluid, the fluid vessel may be a capillary tube. Optimally, the vessel contains discontinuities in fluid, e.g., bubbles, when more than one cell and/or reagent is present, such that separation is achieved between discharging each cell or reagent.

The sequential loading of the vessel with fluid that contains different living cells and/or reagents may be carried out using manual or automated fluid handling devices. For example, the wells in microtiter well plates having 96, 384, or 1536 wells may each contain a different cell, reagent, or reagent concentration. A quantity of fluid may be withdrawn from each well and loaded in sequence into the inlet opening of a capillary tube. Pressure may then be applied to the inlet opening through any of a number of pressure-generating means (e.g., syringe, micropump, etc.) in order to eject a stream of fluid containing the desired cell or reagent.

For simultaneous introduction, the optional reagent may be in the form of a solid or semisolid, and may consist essentially of the reagent as a coating, a pressed pellet, or other solid form that is situated on, for example, an area of the substrate located upstream from the target region. In addition, solid or non-solid reagents may be compounded with an additional material that serves as a binder to form a matrix adapted to controllably release reagent into a carrier upon contact. In such a case, the binder material may swell or be solvated by the carrier to release the reagent into the carrier fluid. When the carrier fluid is aqueous, the binder material may be collagenic or another type of hydrophilic substance, such as a hydrophilic polymer. Suitable hydrophilic polymers include, for example: polyalkyleneoxides, such as PEG and polypropylene glycol (PPG); polyvinylpyrrolidones; polyvinylmethylethers; polyacrylamides, such as, polymethacrylamides, polydimethylacrylamides, and polyhydroxypropylmethacrylamides; polyhydroxyethyl acrylates; polyhydroxypropyl methacrylates; polymethyloxazolines; polyethyloxazolines; polyhydroxyethyloxazolines; polyhydroxypropyloxazolines; polyvinyl alcohols; polyphosphazenes; poly(hydroxyalkylcarboxylic acids); polyoxazolidines; polyaspartamide; polymers of sialic acid (polysialics); copolymers thereof, and mixtures of any of the foregoing. Such hydrophilic materials may be additionally compounded with a hydrophobic material, such as a wax or petroleum jelly, to slow the release of the reagent in contact with an aqueous carrier.

The reagent and the binder material may be provided in an appropriate ratio to release the reagent at a constant rate. When the binder material is polymeric, such as one listed supra, the molecular weight of the binder polymer may be selected according to the desired reagent release rate. Typically, higher molecular weight polymers will result in a slower release rate. In addition, it is preferred that the binder material be substantially immobile with respect to the substrate, to avoid release of the binder material downstream if the binder material will interfere with the function of the reagent or a particular assay being conducted. For example, if the binder material has a potential to interfere with the results of an interaction between cells, it is preferred that the binder material not be released into the fluid. Thus, to avoid binder material being released into the fluid, the binder material may, for example, be covalently bound to the substrate surface. In some instances, the binder material may be appropriate as both a binder material for the reagent as well as a material used to immobilize cells. For example, collagenic materials may both immobilize cells onto the substrate, as well as assist in controlling the release of the optional reagent into the carrier fluid.

As discussed above, the flow passage is typically defined in part by a cover plate that opposes the target region of the substrate surface. Often, the cover plate surface is parallel to the target region of the substrate surface. Similarly, it is preferred that the flow passage of the device is constructed as a conduit. Accordingly, the flow passage is typically defined by opposing sidewalls in fluid-tight contact with the substrate. In some instances, the sidewalls represent an integral portion of the substrate. When the flow passage is a conduit having a constant cross-sectional shape and area, formed lanes are substantially parallel to each other, as well as to the conduit walls. One skilled in the art will recognize that lanes may be narrowed if the conduit is narrowed.

Similarly, the optional cover plate and substrate surfaces may or may not be parallel to each other. Since cells and fluids that may be employed with the invention can be rare or expensive, it is preferred that as small a quantity as possible of cells and fluid be used to flow over the target region as is practicable. However, fluid flow depends on the volume of fluid, as well as the volume of the flow passage. Typically, when the substrate and cover plate surfaces are parallel to each other, the surfaces are located from about 1 μm to about 500 μm from each other. Preferably, the substrate and cover plate surfaces are located from about 20 μm to about 100 μm from each other.

For any of the embodiments described above, it is preferred that the device be constructed in a modular manner to ensure the interchangeability of the components. In particular, certain components may be formed from stock items to lower the cost of the device and to make it cost effective to treat at least the stock components as disposable. For example, as discussed above, the substrate may comprise a glass slide as found in most laboratories and available commercially from, for example, Sigma-Aldrich Corp, St. Louis, Mo. (product number S 8902). Similarly, to facilitate handling, the components of the inventive device may be detachable from each other. As access to the target region of the substrate is limited when it is in an opposing relationship to the cover plate, it is preferred that the substrate be detachable from the cover plate. When the substrate is a detachable and disposable item, such as glass slide, complex capillary tube attachment procedures, which are required before each use of the device, may be avoided if the tubes are essentially permanently connected to the inlets.

The device may be adapted to form cell-containing and reagent-containing streams from fluids of virtually any type, depending on the intended assay. Thus, the fluid may be aqueous and/nor nonaqueous. Nonaqueous fluids include, for example, organic solvents and lipidic liquids.

Since laminar fluid flow is a function of a number of variables, including the geometry of the surfaces over which the fluid flows, flow velocity, and fluid properties (e.g., viscosity), it is important that fluid movement in the inventive device be precisely controlled. Inlets through which fluids containing cells or reagents are introduced into the flow passage typically have a cross-sectional area of 1×10⁻⁵ mm² to about 1 mm², preferably about 5×10⁻⁴ mm² to about 0.1 mm², and optimally 1×10⁻³ mm² to about 1×10⁻² mm². The inlets may have a variety of shapes including, but not limited to, circular, elliptical, square, rectangular, and triangular.

In order to ensure that laminar flow is exhibited in the fluid flowing through the flow passage, a pump is employed to deliver appropriate fluid from a fluid source through the appropriate inlet. Typically, high precision microsyringe pumps are employed to provide fluid flow through capillaries to the inlets. However, other types of pumps may be employed as well. In some instances, one pump is sufficient to provide a motive force to ensure proper fluid flow.

It should be noted that a fluid exhibiting laminar flow over the target region may be employed to attach moieties, e.g., analytes, reagents, cells, and so forth, to a desired area on the substrate. That is, fluid flowing over a desired area on the substrate delivers the moiety to the desired area, thereby allowing for attachment of the moiety to the substrate. The moiety may be attached to the substrate based on techniques known to those skilled in the art, e.g., using a functionalized substrate or a substrate that exhibits surface modification. For example, antibodies may be bound to the substrate so that proteins that contact the bound antibodies immobilize the proteins. Optimally, binding of moieties to the substrate is covalent in nature, although other types of binding, e.g., ionic, hydrogen, and so forth, may also be used.

The method further involves, after the second living cell is placed in contact or proximity with the first living cell (or tissue section containing living cells), detecting a cell-cell interaction, if present, as a result of the contact or proximity between the first living cell and the second living cell. Detectable cell-cell interactions include binding, signal transmission, cell capture, rolling, arrest, adhesion, and diapedesis. A cell-cell interaction may not require direct contact between two cells per se. For example, proximity between two cells may be enough to result in a cell-cell interaction when, for example, one cell releases a cytokine that causes a second cell to react. Proximity in this context is a distance sufficient to effect a cell-cell interaction and includes distances of about 50 microns or less, preferably about 30 microns or less, with distances of 15 microns or less being most preferred. All distances are based on the closest distance between the closest points on the surface of each cell.

The cell-cell interaction may take place between any parts of the cells. For example, surface ligands, generally glycoproteins, located on the cellular surface may facilitate adhesion of one cell to another. These ligands are called cell adhesion proteins and may mediate the adhesion of one cell to another. Membrane fusion events may occur between the lipid components of the cell membrane of two cells, thereby mediating adherence. Such fusion can be facilitated by proteins expressed in either the first living cell or the second living cell. Diapedesis, or the movement or passage of blood-borne cells, especially leukocytes or metastatic tumor cells, through intact capillary walls may be a detectable cell-cell interaction when the first living cell comprises a monolayer or tissue sample of endothelial cells. Signal transmission between lymphocytes, e.g., antigen presentation, may be detected. Of course, additional specific examples of cell-cell interactions that can be detected are well-known to those skilled in the art.

Optionally, the cells may be stained in order to facilitate identification and visualization. Suitable dyes include Malachite green, Sudan black, Coomassie blue, and hematoxylin, among others. Generally, staining a cell is preferred over attaching a label to the cell surface, as the latter may interfere with the cell-cell interaction of interest.

Labels may, of course, be used in order to facilitate detection of the cell and/or the cell-cell interaction. Preferred labels will exist solely in the cytoplasm of the cell. Conventional labels may be introduced into the cell via cellular uptake of labeled moieties, e.g., radiolabled oligonucleotides or binding of a labeled antibody to an exposed cell-surface protein. Cells may be labeled by introduction of naturally fluorescent proteins such as Green Fluorescent Protein (GFP) and mutants of this protein using standard molecular biology techniques known in the art. Many of these labeled moieties are commercially available. In addition, a moiety may be labeled using conventional labeling techniques, such as coupling the moiety with a commercially available activated label, e.g., fluorescein isothiocyanate. Other labeling techniques known in the art may also be used.

The label may be a radioactive label, e.g., ³H, ¹³C, ¹⁴C, ³²P, ¹²⁵I, ¹³¹I, ³⁵S, or ¹⁵N, a fluorescer (e.g., fluorescein and its fluorescent derivatives, phycoerythrin, allo-phycocyanin, phycocyanin, rhodamine, or Texas Red), a chemiluminescer, a photosensitizer, or an enzyme, enzyme substrate, or affinity label (e.g., biotin, peroxidase, or alkaline phosphatase). Scintillation counters, gamma counters, autoradiographers, films, nuclear magnetic resonance (NMR) devices, infrared (IR) detectors, fluorimeters, luminometers, or spectrophotometers are able to detect these or other labels. Of course, some labels may require the addition of a second moiety, e.g., substrate, enzyme or binding partner, for facile detection.

Detectors such as an optical imaging system or a microscope can detect cell-cell interactions. Other detectors include, for example, chromatographic devices, mass spectrometers, immunoassays, fluorescence detectors, and combinations thereof. In addition, any combination of detectors and/or labels may be used for detecting the cell-cell interaction.

A means for detecting a change is used to detect a cell-cell interaction, if present. Such a means may advantageously be a part of the devices described herein, thereby providing a single apparatus for facile testing. While the detecting means will vary depending upon the assay being conducted and the potential signal being produced, one skilled in the art will readily identify those detectors suitable for any particular assay and signal. Furthermore, detecting the interaction may be conducted by inspecting the cells directly on the substrate or by removing the cells, e.g., by scraping the slide. Also, the fluid outflow may be assayed, as once-immobilized cells or bound cells become dislodged as a result of the cell-cell interaction or some other cause.

Cells may become dislodged depending on the rate of flow of the fluid passing over the cells. Thus, another advantage of the present method and invention is that the flow rate of the carrier fluid may be increased to determine what effect increased shear will have on cell-cell interactions. In this way, it is possible to determine not only the strength of the interaction, e.g., binding or adhesion, but to also the specificity of the interaction.

Preferably, the carrier fluid, including any other fluid flowing in the flow passage, comprises a culture medium or perfusion solution, e.g., Hank's balanced salt solution (HBSS), for sustaining the viability of the cells, in addition to providing directionality to the stream of fluid containing the reagent. It must be noted, however, that the fluid or fluids flowing through the flow passage do not necessarily ensure that the cell remains living, although living cells are preferred. Thus, for example, the fluid or fluids may be provided to keep living cells viable in the absence of a toxic reagent. If a toxic reagent is introduced into the fluid or fluids during the assay, cell death may result notwithstanding the presence of the culture medium or perfusion solution.

Culture media suitable for the cells immobilized on the substrate are known to those skilled in the art and are available commercially from, for example, Sigma Inc., St. Louis, Mo. Generally, such media contain mixtures of salts, amino acids, vitamins, nutrients, and other substances necessary to maintain cell health. Preferred salts in the culture medium include, without limitation, NaCl, KCl, NaH₂PO₄, NaHCO₃, CaCl₂, MgCl₂, and combinations thereof. Preferred amino acids are the naturally occurring L-amino acids, particularly arginine, cysteine, glutamine, histidine, isoleucine, leucine, lysine, methionine, phenylalanine, threonine, tryptophan, tyrosine, valine, and combinations thereof. Preferred vitamins in the cell culture include, for example, biotin, choline, folate, nicotinamide, pantothenate, pyridoxal, thiamine, riboflavin, and combinations thereof. Glucose and/or serum, e.g., horse serum or calf serum, are also preferred components of the culture medium. Optionally, antibiotic agents, such as penicillin and streptomycin, may be added to suppress the growth of bacteria. Preferably, the culture medium will contain one or more protein growth factors specific for a particular cell type. For example, many nerve cells require trace amounts of nerve growth factor (NGF) to sustain their viability. Similarly, the culture medium may contain hepatocyte growth factor (HGF) when hepatocytes are present in the assay. Those skilled in the art routinely consider these and other factors in determining a suitable culture medium for any given cell type.

In addition, the carrier fluid or other fluid flowing through the flow passage may contain buffers or perfusion solutions such as Hank's balanced salt solution, with or without the culture medium. Those of skill in the art will recognize other suitable buffers and solutions for use with the present methods and devices.

Nearly any type of cell may be used with the present methods, including eukaryotic, yeast, prokaryotic, and bacterial cells. Preferably, however, the cell is a mammalian cell, e.g., a human cell. Preferred cell types are selected from the group consisting of liver cells, gastrointestinal cells, epithelial cells, endothelial cells, kidney cells, cancer cells, blood cells, stem cells, bone cells, smooth muscle cells, striated muscle cells, cardiac muscle cells, and nerve cells. The cells may originate from a cell line, or may be primary cells.

Particularly preferred cells include endothelial cells (generally as the first living cell) and blood cells (generally as the second living cell). Among blood cells, leukocytes, lymphocytes, red blood cells, and platelets are preferred. Preferred leukocytes include those selected from the group consisting of neutrophils, lymphocytes, monocytes, eosinophils, basophils, and macrophages.

The first living cell and second living cell may comprise a plurality of first living cells and second living cells, respectively. Typically the total number of cells used in any one assay will be from about 2 cells to about 5,000 cells, more preferably from about 2 cells to about 1,000 cells, and most preferably from about 2 cells to about 500 cells.

Typically, immobilized cells are present on the target region as a confluent or subconfluent monolayer within each test lane. The monolayer within the target region may be substantially contiguous or comprise an array of features, each feature comprising at least one cell. The cells may be immobilized onto the solid surface using conventional techniques known to those skilled in the art. For example, the cells may be immobilized by simply contacting the cells to the target region. Areas where a cell is not desired may be protected with a covering, e.g., adhesive tape, which is removed once all cells have been added to the substrate. Cells that may already be located on the substrate may be protected by, for example, cover slips suitably shaped to protect the area containing the immobilized cells. Optionally, a centrifuge may be used. Generally, the force required to immobilize the cell on the target region is from about 200×g to about 500×g.

Alternatively, the surface may be coated with a layer of a cell-adhering substance such as any biological molecule that can facilitate attachment of a living cell. Examples of such substances include collagen, alginate, agar, or other material to immobilize the cells. Preferably the cell-adhering substance is shaped to provide a desired pattern, when present, on the target region. For example, when immobilization of cells in a contiguous layer within the target region is desired, the cell-adhering substance may be contiguously coated onto the target region. However, when it is desirable to provide an immobilized array of cells, the cell-adhering substance may be present as an array of features on the target region. That is, an array of locations on the target region may be coated with an appropriate material to form an array, e.g., checkerboard, spots or other pattern, so that cells may be spatially arranged at specific locations on the solid surface. See, e.g., U.S. Pat. Nos. 5,976,826 and 5,776,748 to Singhvi. In some instances, a photolithographic technique may be employed. U.S. Pat. Nos. 5,202,227 and 5,593,814, each to Matsuda et al., describe a process for preparing a cell arrangement control device wherein a photosensitive, cell-nonadhesive polymer is applied to a cell adhesive surface. The resulting photosensitive cell-nonadhesive polymer layer is irradiated patternwise and developed to leave the irradiated portion on the cell adhesive surface, thereby providing a pattern of the cell-nonadhesive polymer on the cell adhesive surface. As a result, a biological cell culture device may include a surface pattern having a cell adhesive portion and a cell-nonadhesive portion, wherein the cell-nonadhesive portion is covalently bound to the cell adhesive surface. As will be appreciated, such arrays provide for the ability to conduct multiplex assays, i.e., the ability to perform several different experiments on one substrate using different cell types and/or reagents.

The cells may be present on the target region as a tissue section. Immobilization of tissue sections containing cells of interest may be accomplished by first freezing, e.g., to about −15° C. to about −20° C., a relatively large section of tissue. Thereafter, a knife, microtome, or similar sectioning device is used to slice the frozen tissue into sections of desired shape, e.g., lanes. Next, a single section of the tissue is placed onto the target region, e.g., a glass slide, and the section is allowed to “melt” on the target region, thereby immobilizing the cells in the tissue onto the target region. Those skilled in the art will recognize other immobilization techniques that can be used as well.

Thus, the methods and devices described herein are useful for detecting a cell-cell interaction. The methods and devices further provide a means to detect such cell-cell interactions in the presence of a reagent, thereby allowing for the ability to determine the influence of the reagent on a cell-cell interaction. The methods and devices use relatively small amounts of cells and/or reagents, accurately mimic in vivo conditions, and provide for the ability to conduct high-throughput screening.

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of the analytical industry and the like, which are within the skill of the art. Such techniques are explained fully in the literature.

In the following examples, efforts have been made to ensure accuracy with respect to numbers used (e.g., amounts, temperature, etc.) but some experimental error and deviation should be accounted for. Unless indicated otherwise, temperature is in degrees Celsius and pressure is at or near atmospheric. All reagents were obtained commercially unless otherwise indicated.

### Example 1

T-Cells (Jurkat cell clone) were flowed over and allowed to adhere to 24-hour treated tumor necrosis factor-alpha (TNF) primary human endothelial cells (HUVECs). Briefly, HUVEC's (trypsinized, if starting with a monolayer) were plated into a two well Biocoat slide. The cells were then cultured in media to which a 10% fetal calf serum (FCS) solution was added. A sufficient amount of time was allowed to pass until the cells reached confluency. HUVECs were then treated with TNF (100 μg/ml) for 24 hours. On the following day, a 1 ml aliquot of Jurkat cells was treated with Calcein-AM (1-4 μM). After one hour, Calcein-AM (to 1-4 μM) was again added and allowed to remain for 30 minutes. A stage heater was then turned on and set for 37° C. The HUVEC-containing slide was placed into the appropriate docking station in the flow passage, which was then purged with HBSS (Hank's balanced saline solution). The cell monolayer on the slide was visualized using light microscopy. The Jurkat cell aggregates were resuspended/sheared using a yellow pipette tip (repeated five times). Two hundred μ of Calcein-labeled Jurkat cells were loaded into a microtiter well from which an autosampler pump was programmed to deliver fluid at 0.02 μl/sec into the flow passage. Laminar flow was maintained at all times for fluids flowing through the flow passage. The loading of cells was visualized, paying particular attention to photobleaching. The cells were loaded for one minute, followed by a no-loading period of 1-10 minutes to allow cell binding. A “cell-maintenance” program was initiated in the autosampler pump to provide a flow rate of 0.1 μl/sec. Thereafter, the flow rate was sequentially increased to flow rates of 0.5, 1, 2, 4, and 8 μl/sec. Each flow rate was maintained for 0.5-5 minutes, generally about one minute. Adhering cells were visualized, again paying particular attention to photobleaching. A maximum flow rate was run for an additional 3-10 minutes, followed by quantitation of the remaining bound fluorescent cells from negative images.

### EXAMPLE 2

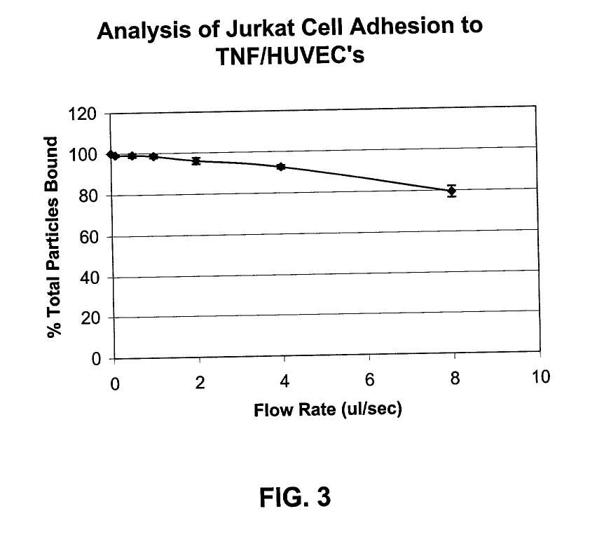

The procedure of Example 1 was adapted to test the strength of binding. The Jurkat cells were exposed to increasing shear stress by increasing the flow rate up to 8 μl/sec. The number of cells that remained bound was quantified after each increase in flow rate. As seen in FIG. 3, Jurkat cells remained strongly adhered to the TNF-treated HUVEC monolayer.

### EXAMPLE 3

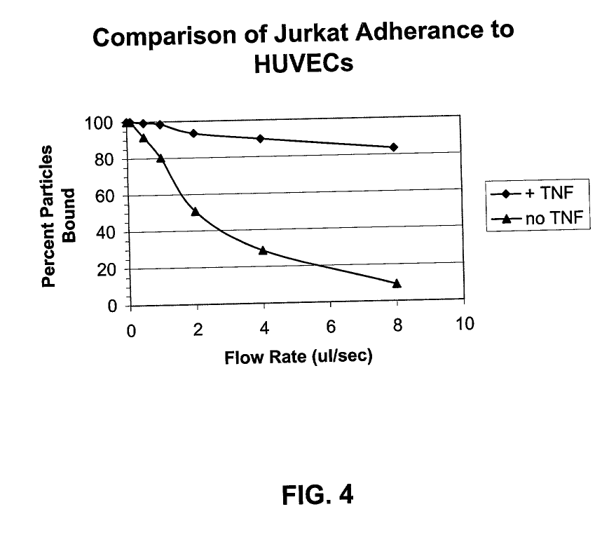

The procedure of Example 1 was adapted to test binding strength and specificity. Jurkat cells were flowed over and allowed to adhere to untreated (no TNF) or 24-hour TNF treated (+TNF) HUVECs. Thereafter, cell adherence was tested with an increasing shear stress by increasing the flow rate over the cells. As seen in FIG. 4, more Jurkat cells remained adhered after increasing the flow rate to 8 μl/sec when flowed over +TNF HUVECs, indicating that TNF induces an increase in the adherence profile of Jurkat cells to endothelial cells.

### EXAMPLE 4

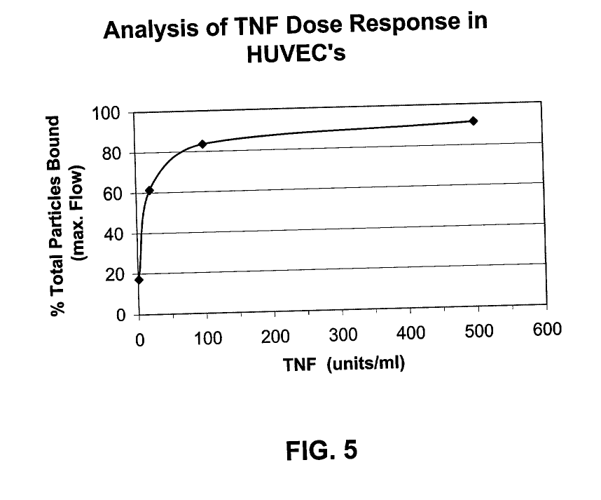

The procedure of Example 1 was adapted to test binding strength and specificity. Jurkat cells were flowed over and allowed to adhere to HUVECs treated with various concentrations of TNF, ranging from 0 to 500 units/ml. Thereafter, cell adherence was tested against increasing shear stress by increasing the flow rate over the cells. Percent adherence was determined at the maximum flow rate of 8 μl/sec. As seen in FIG. 5, Jurkat cell adherence increased as a function of the TNF concentration used to treat the HUVECs, indicating that such TNF treatment induces the adherence of Jurkat cells to endothelial cells.

### EXAMPLE 5

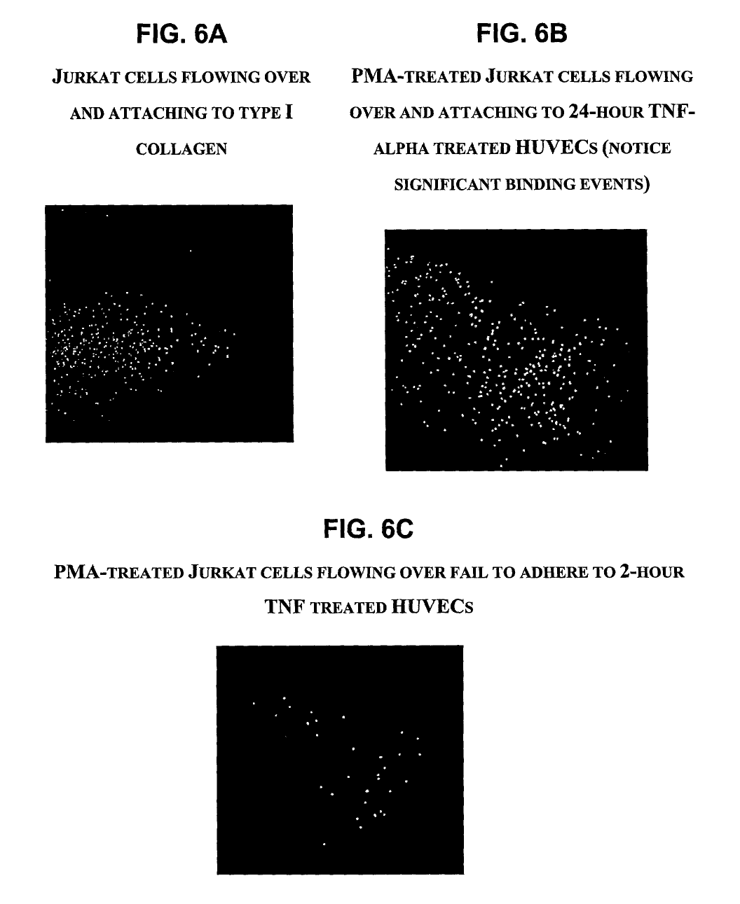

The procedure of Example 1 was adapted for the following tests. As seen in FIG. 6A, Jurkat cells were introduced over and onto a substrate having type I collagen attached thereto, thereby allowing for the attachment of the Jurkat cells to the collagen. As seen in FIG. 6B, PMA-treated Jurkat cells, i.e., Jurkat cells activated via incubation in a phorbol 12-myristate 13-acetate (PMA) solution (10 μg/ml in dimethylsulfoxide), adhere to 24 hour TNF-treated HUVECs. As seen in FIG. 6C, however, significantly fewer PMA-treated Jurkat cells adhere to HUVECs that have only been exposed to a two-hour treatment of TNF.

We claim:

1. A method for detecting a cell-cell interaction comprising:
(a) providing a flow passage defined at least in part by a substrate having a first living cell immobilized on a surface thereof;
(b) introducing a second living cell by controlled delivery of a carrier fluid containing the second living cell in contiguous laminar flow through the flow passage, thereby effecting contact or proximity between the first living cell and second living cell; and
(c) detecting a cell-cell interaction, if present, as a result of the contact or proximity between the first living cell and second living cell.

2. The method of claim 1, wherein step (a) comprises placing the substrate surface in opposing relationship to a cover plate to further define the flow passage.

3. The method of claim 2, wherein step (a) comprises placing the substrate surface in fluid-tight contact relationship with opposing sidewalls to further define the flow passage.

4. The method of claim 1, wherein the first living cell is immobilized prior to step (a).

5. The method of claim 1, wherein the first living cell is immobilized after step (a).

6. The method of claim 5, wherein the first living cell is placed and immobilized on the substrate surface through use of laminar flow cellular delivery.

7. The method of claim 6, wherein the substrate surface comprises a cell-adhering site.

8. The method of claim 7, wherein the cell-adhering site comprises a biological material that facilitates attachment of a living cell.

9. The method of claim 8, wherein the biological material is collagen.

10. The method of claim 1, comprising a plurality of first living cells.

11. The method of claim 10, wherein the plurality of first living cells is immobilized as a monolayer on the substrate surface.

12. The method of claim 10, wherein the plurality of first living cells is immobilized as a subconfluent layer on the substrate surface.

13. The method of claim 10, wherein the plurality of first living cells is in the form of a tissue section.

14. The method of claim 1, wherein the first living cell, the second living cell, or both are primary cells.

15. The method of claim 14, wherein the primary cells are mammalian, yeast, prokaryotic or bacterial.

16. The method of claim 1, wherein the first living cell and second living cell are independently selected from the group consisting of liver cells, gastrointestinal cells, epithelial cells, endothelial cells, kidney cells, cancer cells, blood cells, stem cells, bone cells, smooth muscle cells, striated muscle cells, cardiac muscle cells, and nerve cells.

17. The method of claim 1, wherein the first living cell is an endothelial cell.

18. The method of claim 1, wherein the second living cell is a blood cell.

19. The method of claim 18, wherein the blood cell is a leukocyte.

20. The method of claim 19, wherein the leukocyte is selected from the group consisting of neutrophils, lymphocytes, monocytes, eosinophils, basophils, and macrophages.

21. The method of claim 18, wherein the blood cell is a lymphocyte.

22. The method of claim 18, wherein the blood cell is a red blood cell.

23. The method of claim 1, wherein the substrate is made from glass.

24. The method of claim 1, wherein the carrier fluid comprises a medium appropriate to sustain living cells.

25. The method of claim 1, wherein the detected cell-cell interaction is selected from the group consisting of binding, signal transmission, cell capture, rolling, arrest, adhesion, and diapedesis.

26. The method of claim 21, wherein the detected cell-cell interaction is binding.

27. The method of claim 26, further comprising increasing the flow rate of the carrier fluid and determining whether binding between the cells is maintained at the increased flow rate.

28. The method of claim 1, further comprising before step (b), introducing a reagent into the carrier fluid, thereby effecting contact or proximity between the reagent and the first living cell, second living cell or both.

29. The method of claim 28, wherein the reagent is selected from the group consisting of a small drug molecule, amino acid, amino acid analog, peptide, protein, nucleotide, nucleoside, oligonucleotide, antibody, and conjugates thereof

30. The method of claim 1, further comprising after step (b), introducing a reagent into the carrier fluid, thereby effecting contact or proximity between the reagent and the first living cell, second living cell, or both.

31. The method of claim 30, wherein the reagent is selected from the group consisting of a small drug molecule, amino acid, amino acid analog, peptide, protein, nucleotide, nucleoside, oligonucleotide, antibody, and conjugates thereof.

32. The method of claim 1, further comprising simultaneously with step (b), introducing a reagent into the carrier fluid, thereby effecting contact or proximity between the reagent and the first living cell, second living cell or both.

33. The method of claim 32, wherein the reagent is selected from the group consisting of a small drug molecule, amino acid, amino acid analog, peptide, protein, nucleotide, nucleoside, oligonucleotide, antibody, and conjugates thereof.

34. The method of claim 1, wherein the total number of cells used in the method is from about 2 to about 5,000 cells.

35. The method of claim 34, wherein the total number of cells used in the method is from about 2 to about 1,000 cells.

36. The method of claim 35, wherein the total number of cells used in the method is from about 2 to about 500 cells.

37. The method of claim 1, wherein contact between the first living cell and second living cell is established.

38. The method of claim 1, wherein proximity between the first living cell and second living cell is established.

39. The method of claim 38, wherein the proximity between the first living cell and the second living cell is about 50 microns or less.

40. The method of claim 1, wherein the second living cell is introduced via hydrodynamically focused flow.

41. A device for providing a cell-cell interaction, comprising:
(a) a substrate having a first living cell immobilized on a surface thereof;
(b) at least one inlet for introducing a carrier fluid containing a second living cell;
(c) a means for controlling delivery of the carrier fluid in contiguous laminar flow so as to enable contact or proximity between the first living cell and the second living cell; and
(d) at least one outlet enabling removal of fluid from the device.

42. The device of claim 41, further comprising a detecting means for detecting a cell-cell interaction, if present, resulting from the contact or proximity between the first living cell and the second living cell.

43. The device of claim 42, wherein the detecting means is a microscope.

44. The device of claim 41, wherein the flow passage is further defined by a cover plate having a surface that opposes the surface of the substrate.

45. The device of claim 41, wherein the substrate is detachable.

46. The device of claim 41, wherein the substrate is substantially planar.

47. The device of claim 44, wherein the substrate and cover plate surfaces are substantially planar.

48. The device of claim 44, wherein the substrate and cover plate surfaces are located from about 1 μm to about 500 μm from each other.

49. The device of claim 48, wherein the substrate and cover plate surfaces are located from about 20 μm to about 100 μm from each other.

50. The device of claim 41, wherein the flow passage is further defined by opposing sidewalls in fluid-tight contact with the substrate.

51. The device of claim 50, wherein the sidewalls are substantially parallel to each other.

52. The device of claim 41, wherein the means for controlling delivery of the carrier fluid in contiguous laminar flow is adapted to provide constant velocity flow.

53. The device of claim 41, further providing an additional inlet for the introduction of a stream of reagent into the carrier fluid upstream from the first living cell.

54. The device of claim 41, wherein the first living cell comprises a plurality of first living cells.

55. The device of claim 41, wherein the second living cell comprises a plurality of second living cells.

56. The device of claim 41, wherein the substrate surface comprises a cell-adhering site.

57. The device of claim 56, wherein the cell-adhering site comprises a biological material that facilitates attachment of a living cell.

58. The device of claim 57, wherein the biological material is collagen.

59. The device of claim 41, wherein the means for controlling delivery of the carrier fluid comprises two guide stream inlets for introducing guide streams, and further wherein the carrier fluid inlet and the guide stream inlets are positioned such that the carrier fluid is interposed between the guide streams.

60. The device of claim 59, wherein the means for controlling delivery of the carrier fluid further comprises means for controlling flow rates of the guide streams such that the carrier fluid is hydrodynamically focused.
